SEQUENCE LISTING (8 sequences)

SEQ ID NO: 1 - protein, 179 aa, Oryza sativa
  PEPTIDE (1)..(179)
MAGSGRDRDPLVVGRVVGDVLDAFVRSTNLKVTYGSKTVSNGCELKPSMVTHQPRVEVGGNDMRTFYTLVMVDPDAPSPSDPNLREYLHWLVTDIPGTTAASFGQEVMCYESPRPTMGIHRLVFVLFQQLGRQTVYAPGWRQNFNTKDFAELYNLGSPVAAVYFNCQREAGSGGRRVYN

SEQ ID NO: 2 - protein, 179 aa, Artificial Sequence
  synthetic FT protein sequence not identical to native plant proteins
  PEPTIDE (1)..(179)
MAGSGRDREPLVVGRVVGDVLDPFVRTTNLRVTYGSRTVSNGCELKPSMVTHQPRVEVGGNDMRTFYTLVMVDPDAPSPSDPNLREYLHWLVTDIPGTTGASFGQEVMCYESPRPTMGIHRFVLVLFQQLGRQTVYAPGWRQNFNTRDFAELYNLGSPVAAVYFNCQREAGSGGRRMYN

SEQ ID NO: 3 - DNA, 568 bp, Artificial Sequence
  synthetic coding region for synthetic FT protein
  5′UTR (1)..(16)
  CDS (17)..(556)
  3′UTR (557)..(568)
acacagcgctaccaccatggcgggaagcggccgcgaccgcgagccactcgtcgtgggacgcgtggtcggcgacgtcctcgacccgttcgtgcgcacgacgaacctgcgcgtcacctacggcagccgcaccgtctccaacggatgcgagctgaagccctccatggtgacccaccagccgcgcgtcgaggtcggagggaacgacatgcgcacattctacaccctggtcatggtcgaccccgacgcacccagtccgagcgacccgaacctgcgcgagtacctgcactggctggtcaccgacatcccaggcaccaccggagccagcttcggccaggaggtcatgtgctacgagtccccacgcccgactatgggcatccacaggttcgtcctggtcctgttccagcagctgggacgccagaccgtctacgcaccaggctggaggcagaacttcaacacccgcgacttcgcagagctgtacaacctgggaagcccggtggcagcggtgtacttcaattgccagagggaggcagggagcggagggcgcagaatgtacaactgaggcgcgccaacc

SEQ ID NO: 4 - protein, 179 aa, Artificial Sequence; identical to SEQ ID NO: 2
  Synthetic Construct

SEQ ID NO: 5 - DNA, 6950 bp, Artificial sequence
  gene region of plasmid piFT SEQ ID No 4
  promoter (1)..(426): CaMV 35S promoter
  Intron (427)..(1237): maize hsp70 intron
  CDS (1247)..(2770): GFP-NPTII fusion protein
  terminator (2771)..(2970): T35S terminator
  promoter (2971)..(3589): FMV promoter
  CDS (3602)..(5074): chimeric XEV CDS (X = LexA binding, E = Estrdiol ligand binding domain, V = VP16)
  terminator (5075)..(5348): PinII terminator
  protein_bind (5349)..(5524): LexA binding sites
  promoter (5525)..(5582): minimal CaMV35S promoter
  Intron (5583)..(6055): Adh1 intron
  CDS (6077)..(6616): synthetic FT CDS
  terminator (6617)..(6950): NOS3′ terminator
atggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctctcgacaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggctcgagcacaccaccatgagcaagggcgaggagctgttcactggggtggtgcccatcctggtcgagctggacggcgacgtgaacggccacaagttcagcgtcagcggcgagggcgagggcgacgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgagccgaggtgaagttcgagggcgacaccctggtgaaccgcatcgagctgaagggcatcgacttcaaggaggacggcaacatcctggggcacaagctggagtacaactacaacagccacaacgtctacatcatggctgacaagcagaagaacggcatcaaggtcaacttcaagatccgccacaacatcgaggacggcagcgtccagctcgccgaccactaccagcagaacacgcccatcggcgacggtcccgtgctgctgcccgacaaccactacctgagcacccagtccgctctgagcaaggaccccaacgagaagcgcgaccacatggtcctgctggagttcgtcaccgcagctggcatcacccacggcatggacgagctgtacaaggctaccggaggaagcatgatcgagcaggacggcctgcacgctggctccccagctgcctgggtggagaggctgttcggctacgactgggctcagcagaccatcggctgctccgacgctgccgtgttcaggctgtccgcacagggcaggccagtgctgttcgtgaagaccgacctgtccggagccctgaacgagctccaggacgaggcagccaggctgtcctggctggccaccaccggagtgccgtgcgcagccgtgctggacgtggtgaccgaggcaggcagggactggctgctgctgggcgaggtgccaggccaggacctgctgtcctcccacctggcaccggcagagaaggtgtccatcatggccgacgccatgaggaggctgcacaccctggacccagccacctgcccgttcgaccaccaggccaagcacaggatcgagagggccaggaccaggatggaggcaggcctggtggaccaggacgacctggacgaggagcaccagggcctggcaccagccgagctgttcgccaggctgaaggccaggatgccggacggcgaggacctggtggtgacccacggcgacgcctgcctgccgaacatcatggtggagaacggcaggttctccggcttcatcgactgcggcaggctgggcgtggccgaccgctaccaggacatcgccctggccaccagggacatcgccgaggagctgggaggcgagtgggcagacaggttcctggtgctgtacggcatcgcagcaccggactcccagaggatcgccttctaccgcctgctggacgagttcttctgaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtcctgcaggactactgcgatcgctcatcaaaatatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactaaccaatatttctcacgcgtaccaccatgggcaaggccctgaccgccaggcagcaggaggtgttcgacctgatcagggaccacatctcccagaccggcatgccaccgaccagggccgagatcgcccagaggctgggcttcaggtccccgaacgcagccgaggagcacctgaaggccctggccaggaagggcgtgatcgagatcgtgtccggagcctccaggggcatcaggctgctccaggaggaagaggaaggcctgccgctggtgggcagggtggccgcaggcgagccgtccagcggaggcgacccgtccgcaggcgacatgagggcagccaacctgtggccgagcccgctgatgatcaagaggagcaagaagaacagcctggccctgagcctgaccgccgaccagatggtgagcgccctgctggacgccgagccaccgatcctgtactccgagtacgacccgaccaggccgttcagcgaggccagcatgatgggcctgctgaccaacctggccgacagggagctggtgcacatgatcaactgggccaagagggtgccaggcttcgtggacctgaccctgcacgaccaggtgcacctgctggagtgcgcctggctggagatcctgatgatcggcctggtgtggaggagcatggagcacccggtgaagctgctgttcgcaccgaacctgctcctggacaggaaccagggcaagtgcgtggagggcatggtggaaatcttcgacatgctcctggccacctccagcaggttcaggatgatgaacctccagggcgaggagttcgtgtgcctgaagagcatcatcctgctcaacagcggcgtgtacaccttcctgtccagcaccctgaagagcctggaggagaaggaccacatccacagggtgctggacaagatcaccgacaccctgatccacctgatggccaaggcaggcctgaccctccagcagcagcaccagaggctggcccagctgctgctgatcctgagccacatcaggcacatgagcaacaagggcatggagcacctgtactccatgaagtgcaagaacgtggtgccgctgtacgacctgctgctggagatgctggacgcccacaggctgcacgcaccgacctccaggggaggcgcaagcgtggaggagaccgaccagagccacctggccaccgcaggcagcacctccagccacagcctccagaagtactacatcaccggcgaggccgagggcttcccagccaccgtgggaggcagcggagcaccgccaaccgacgtgagcctgggcgacgagctgcacctggacggcgaggacgtggcgatggcccacgccgacgccctggacgacttcgacctggacatgctgggcgacggcgacagcccaggaccgggcttcactccgcacgacagcgcaccgtacggagccctggacatggccgacttcgagttcgagcagatgttcaccgacgccctgggcatcgacgagtacggaggctgacctaggccctagacttgtccatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatattcctggttatatatacagcatatactgtatatatatacagtttatactggttaatcatccagctattcctgtatgatcatacagtaatccctggtattatatccagtaattactgtatgtacatacagttcacactggtttatcatacagctattcctgtatgcgcatacagtatagacccttcctctatataaggaagttcatttcatttggagaggacacgctgaagctagtcgtcgacgaaggtgcaaggattgctggagcgtcaaggatcattggtgtcgacctgaaccccagcagattcgaagaaggtacagtacacacacatatgtatatatgtatgatgtatcccttcgatcgaaggcatgccttggtcgaataactgagtagtcattttattacgttattttgacaagtcagtagttcatccatttgtcccattttttcagctaggaagtttggttacactggccttggtctaataactgagtagtcattttattacgttgtttcgacaagtcagtagctcatccatctgtcccatttttttcagctaggaagtttggttacactggacttggtctaataactgagtagtcattttattacgttgtttcgacaagtcattagctcatccatctgtcccatttttcagctaggaagttcggttgcactgaatttgtgaacccaaaagaccacaacaagccgtcgacaccagcgctaccaccatggcgggaagcggccgcgaccgcgagccactcgtcgtgggacgcgtggtcggcgacgtcctcgacccgttcgtgcgcacgacgaacctgcgcgtcacctacggcagccgcaccgtctccaacggatgcgagctgaagccctccatggtgacccaccagccgcgcgtcgaggtcggagggaacgacatgcgcacattctacaccctggtcatggtcgaccccgacgcacccagtccgagcgacccgaacctgcgcgagtacctgcactggctggtcaccgacatcccaggcaccaccggagccagcttcggccaggaggtcatgtgctacgagtccccacgcccgactatgggcatccacaggttcgtcctggtcctgttccagcagctgggacgccagaccgtctacgcaccaggctggaggcagaacttcaacacccgcgacttcgcagagctgtacaacctgggaagcccggtggcagcggtgtacttcaattgccagagggaggcagggagcggagggcgcagaatgtacaactgaggcgcgcccccgggaatgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtacc

SEQ ID NO: 6 - protein, 507 aa, Artificial sequence
  Synthetic Construct
MSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKATGGSMIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 7 - protein, 490 aa, Artificial sequence
  Synthetic Construct
MGKALTARQQEVFDLIRDHISQTGMPPTRAEIAQRLGFRSPNAAEEHLKALARKGVIEIVSGASRGIRLLQEEEEGLPLVGRVAAGEPSSGGDPSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATVGGSGAPPTDVSLGDELHLDGEDVAMAHADALDDFDLDMLGDGDSPGPGFTPHDSAPYGALDMADFEFEQMFTDALGIDEYGG

SEQ ID NO: 8 - protein, 179 aa, Artificial sequence; identical to SEQ ID NO: 2
  Synthetic Construct

(19) United States
(12) Patent Application Publication
(10) Pub. No.: US 2018/0057831 A1
(43) Pub. Date: Mar. 1, 2018

(54) Inducible flowering for fast generation times in maize and sorghum

(71) Applicant: Michael E. Fromm, Lincoln, NE (US)

(72) Inventor: Michael E. Fromm, Lincoln, NE (US)

(21) Appl. No.: 15/680,341

(22) Filed: Aug. 18, 2017

Related U.S. Application Data
(60) Provisional application No. 62/367,933, filed on Jul. 28, 2016.

(51) Int. Cl. C12N 15/82; C07K 14/415

(52) U.S. Cl. CPC C12N 15/827; C07K 14/415; C12N 15/8238

(57) ABSTRACT

A method of producing faster flowering times in corn and sorghum plants is presented herein. Corn and sorghum plants comprising a non-native flowering gene that flower faster by at least three developmental leaves than control plants are also presented herein.

### CROSS-REFERENCE TO RELATED APPLICATIONS

U.S. Provisional 62/377,924 filed 22 Aug. 2016, incorporated by reference herein in its entirety.

### INCORPORATION OF SEQUENCE LISTING

The sequence listing contained in the file named “Flowering FT CRF_ST25.txt”, which is 41,736 bytes in size (measured in operating system MS-Windows), contains 4 sequences, and is contemporaneously filed with this specification by electronic submission (using the United States Patent Office EFS-Web filing system) and is incorporated herein by reference in its entirety. The information recorded in computer readable form is identical to the written sequence listing and drawings submitted in non-provisional patent application Ser. No. 15/680,341, filed Aug. 18, 2017, and the computer readable submission of sequences includes no new matter.

### STATEMENT REGARDING FEDERALLY SPONSORED RESEARCH OR DEVELOPMENT

Not Applicable.

### BACKGROUND OF THE INVENTION

Expression of flowering protein Flowering Locus T (FT) triggers flowering in many plant species and ectopic overexpression induces early flowering in many species. FT does not work alone and is part of a complex gene network regulating flowering time in most plants where it interacts with its partner FLOWERING LOCUS D (FD; known as DLF1 in corn) in induce flowering genes (For review see Meng et al., 2011, The Plant Cell, Vol. 23: 942-960). Transgenic expression of FT causes early flowering in most plants tested. Fast flowering, fast seed-to-seed generation times are useful for research and plant breeding. Fast flowering types of corn are known, such as Gaspe Flint and Fast-Flowering Mini-Maize (McCaw et al., 2016, Genetics. 116.191726). However, these fast flowering lines are due to complex whole genome genetic differences, and are not due to a single transgene such as occurs when FT is overexpressed in many plant species such as Arabidopsis or wheat. This genetic complexity limits the use of the fast flowering lines in elite commercial germplasm research and breeding.

Two species that appear to be biologically different in the role of their FT genes in flowering are corn (Zea mays L) and sorghum (Sorghum bicolor L). For corn, of the 15 FT candidate homologs in the genome, only six are expressed in leaves (Meng et al., 2011, The Plant Cell, Vol. 23: 942-960) which is where FT is normally expressed before being transported to the meristem. These six (ZCN7, ZCN8, ZCN12, ZCN14, ZCN18, and ZCN26) do not include the closest FT homolog (ZCN15) located at a chromosomal position syntenic with the FT genes of rice (Hd3a/b). ZCN15 is not included as it is not expressed in leaves where normal FT expression occurs.

ZCN8's leaf expression profile makes it a candidate FT (Meng et al., 2011, The Plant Cell, Vol. 23: 942-960). ZCN8 was considered the closest candidate for FT function in corn as ZCN8 interacts with DLF1 while ZCN15 interacted weakly with DLF1 (Meng et al., 2011, The Plant Cell, Vol. 23: 942-960) and overexpression of ZCN8 in Arabidopsis can complement FT function in a ft-1 mutant line of Arabidopsis (Lazakis, et al., 2011, Journal of Experimental Botany, Vol. 62, No. 14, pp. 4833-4842).

Overexpression of ZCN8 in transgenic corn plants slightly affected leaf number (a measure of when flowering occurs) as transgenic plant overexpressing ZCN8 had one to two fewer leaves (17 to 18 leaves) compared to control plants with 19 leaves. Down regulation of ZCN8 with a microRNA increased leaf numbers relative to controls. These results support a role for ZCN8 in controlling flowering in corn (Meng et al., 2011, The Plant Cell, Vol. 23: 942-960). These results suggests control of flowering has evolved differently in corn as the syntenic FT ortholog ZCN15 does not appear to control flowering time in corn and the overexpression of ZCN8, the best candidate for FT function in corn, has only mild effects on flowering time in transgenic plants. In further support of this, the atypical roles of FT in corn appear similar to those of FT in sorghum, a close evolutionary relative of corn (Yang et al., BMC Plant Biology 2014, 14:148; Wolabu et al., 2016, New Phytol. 210(3):946-59).

Seed Visual Markers.

Seed color markers have been used to follow traits, such as a male sterility trait. The red fluorescent protein DsRED has been linked to a male sterility/fertility gene for producing hybrid corn and many fluorescent proteins suitable for seed visual markers are available (Gert-Jan Kremers, Sarah G. Gilbert, Paula J. Cranfill, Michael W. Davidson, David W. Piston J Cell Sci 2011 124: 157-160), operably linked for plant gene expression, and preferably after conversion to a synthetic gene with plant preferred codons.

Regulated Gene Expression.

Genes for some traits are turn on and off either temporarily or permanently. Temporary systems, including but not limited to inducible systems or virus expression systems, are useful to express or repress a gene for a limited period of time. Many types of inducible or repression/activation types of gene regulation systems are known. Inducible DNA methyltransferase fusion protein expression can be with promoters that include, but are not limited to, a PR-la promoter (US Patent Application Publication Number 20020062502) or a GST II promoter (WO 1990/008826 A1). Additional examples of inducible promoters include, without limitation, the Adh1 promoter which is inducible by hypoxia or cold stress, the Hsp70 promoter which is inducible by heat stress, and the PPDK promoter which is inducible by light.

Inducible Chimeric Transcription Factor Systems.

Such transcription factor/promoter systems include, but are not limited to: i) DNA binding-activation domain-ecdysone receptor transcription factors/cognate promoters that can be induced by methoxyfenozide, tebufenozide, and other compounds (US Patent Application Publication Number 20070298499); ii) chimeric tetracycline repressor transcription factors/cognate chimeric promoters that can be repressed or de-repressed with tetracycline (Gatz, C., et al. (1992). Plant J. 2, 397-404), estradiol or dexamethasone inducible promoters (Aoyama and Chua, The Plant Journal (1997) 11(3):605-612; Zuo et al., The Plant Journal (2000) 24(2):265-273), an alcohol inducible AlcR system (U.S. Pat. No. 6,605,754, incorporated by reference herein in its entirety), and the like (for review, see Corrado and Karali, 2009, Biotechnol Adv. 27(6):733-43).

DNA Recombinases.

For more permanent or longer term conditional gene switches, DNA recombinases have been used. Methods based on site-specific recombination systems have been described to remove marker genes or obtain randomly integrated single copy transgenes by excising excess linked copies from the genome (Srivastava and Ow, 1999 Proc. Natl. Acad. Sci. USA, 96:11117-11121; Srivastava and Ow, 2001 Plant Mol. Biol. 46:561-566) and to insert DNA at a known chromosome location in the genome (O'Gorman et al., 1991 Science, 251:1351-55; Baubonis and Sauer, 1993 Nucl., Acids Res., 21:2025-29; Albert et al., 1995 Plant J., 7:649-59). These methods make use of site-specific recombination systems that are freely reversible. These reversible systems include the following: the Cre-lox system from bacteriophage P1 (Baubonis and Sauer, 1993, supra; Albert et al., 1995 Plant J., 7:649-59), the FLP-FRT system of Saccharomyces cerevisiae (O'Gorman et al., 1991, supra), the R-RS system of Zygosaccharomyces rouxii (Onouchi et al., 1995 Mol. Gen. Genet. 247: 653-660), a modified Gin-gix system from bacteriophage Mu (Maeser and Kahmann, 1991 Mol. Gen. Genet., 230:170-76), the .beta.-recombinase-six system from a Bacillus subtilis plasmid (Diaz et al., 1999 J. Biol. Chem. 274: 6634-6640), and the .gamma. .delta.-res system from the bacterial transposon Tn1000 (Schwikardi and Dorge, 2000 FEBS let. 471: 147-150). Cre, FLP, R, Gin, .beta.-recombinase and .gamma. .delta. are the recombinases, and lox, FRT, RS, gix, six and res the respective recombination sites (reviewed by Sadowski, 1993 FASEB J., 7:750-67; Ow and Medberry, 1995 Crit. Rev. Plant Sci. 14: 239-261).

Phloem Active Promoters.

Examples of promoters that direct vascular/phloem gene expression as part of their developmental program include regulatory sequences from viral (Benfey et al., EMBO J. 9[6] 1685-1696, 1990) and bacterial (Kononowicz et al., Plant Cell 4:17-27, 1992) genes as well as plant genes (Liang et al., Proc. Natl. Acad. Sci. USA 86:9284-9288, 1989); Keller and Baumgartner, Plant Cell 3:1051-1061). Transcriptional regulatory sequences have also been isolated from phloem-limited DNA viruses, such as the rice tungro virus (Bhattacharyya-Pakrasi et al., Plant J. 4[1] 71-79, 1993) and the commelina yellow mottle virus (Medberry et al., Plant Cell 4:185-192, 1992), that direct phloem-specific gene expression. In addition, the transcriptional regulatory elements of plant genes encoding proteins that have phloem-associated functions, such as sucrose synthase (Yang and Russell, Proc. Natl. Acad. Sci. USA 87:4144-4148, 1990), glutamine synthetase (Edwards et al., Proc. Natl. Acad. Sci. USA 87:3459-3463, 1990), and a phloem-specific isoform of the plasma membrane H+-ATPase (DeWitt et al., Plant J. 1[1]: 121-128, 1991), have been shown to direct phloem-specific expression of reporter genes in transgenic plants.

Gene Editing of Endogenous Genes

Considerable progress has been made in targeting DNA binding proteins to specific DNA sequences in the genomes of live cells for gene editing, i.e., making specific sequence changes at specific gene targets in the genome of plants. Zinc fingers, TALENS, and CRISPR/CAS9 proteins or protein/RNA complexes are experimentally amenable to changes in their amino acid sequences or RNA targeting sequences to facilitate their binding to specific DNA sequences (Cai and Yang 2014; Carroll 2014; Gersbach and Perez-Pinera 2014; Kim and Kim 2014). Of these, the most convenient method to target a protein to a specific DNA sequence is with the CRISPR/CAS9 protein/RNA complex (Esvelt, Mali et al. 2013; Hou, Zhang et al. 2013; Fonfara, Le Rhun et al. 2014; Hsu, Lander et al. 2014; Sander and Joung 2014). CRISPR proteins are members of a large Cas3 class of helicases found in many prokaryotes [see (Jackson, Lavin et al. 2014) and references therein], herein referred to as CRISPR/CAS9. CRISPR/CAS9 class of proteins bind either a single guide RNA or two annealed RNAs, that target specific DNA sequences through DNA/RNA complementary base pairing, facilitated by the CRIPSR/CAS9 protein unwinding of the DNA (Cai and Yang 2014; Carroll 2014; Gersbach and Perez-Pinera 2014; Kim and Kim 2014). Multiple single guide RNAs (sgRNAs) can be used concurrently, with examples of two (Mao, Zhang et al. 2013), three (Ma, Chang et al. 2014), four (Perez-Pinera, Kocak et al. 2013; Ma, Shen et al. 2014), five (Jao, Wente et al. 2013), six (Liu et al., Insect Biochem Mol Biol. 2014 June; 49:35-42), or seven (Sakuma, Nishikawa et al. 2014). Most designs utilize repeats of an intact sgRNA gene with its own Pol III U6 or U3 promoter (Sakuma, Nishikawa et al. 2014).

The CRISPR/CAS9 system can be used for DNA cleavage, DNA nicking, or binding DNA with a nuclease-inactive form. Predictive software for useful sgRNA designs is available (Bae, Park et al. 2014; Kunne, Swarts et al. 2014; Xiao, Cheng et al. 2014; Xie, Zhang et al. 2014) and progress on the mechanisms of CRISPR DNA recognition is proceeding.

Sequence specific DNA binding proteins such as zinc fingers, TALENS, and CRISPR proteins are useful in plants as well (Belhaj, Chaparro-Garcia et al. 2013; Shan, Wang et al. 2013; Chen and Gao 2014; Fichtner, Urrea Castellanos et al. 2014; Liu and Fan 2014; Lozano-Juste and Cutler 2014; Puchta and Fauser 2014). Recent publications use catalytically active nucleases in Arabidopsis (Jiang, Zhou et al. 2013; Fauser, Schiml et al. 2014; Feng, Mao et al. 2014; Gao and Zhao 2014; Jiang, Yang et al. 2014); or a nickase in Arabidopsis (Fauser, Schiml et al. 2014); maize (Liang, Zhang et al. 2014); rice (Jiang, Zhou et al. 2013; Miao, Guo et al. 2013; Xu, Li et al. 2014; Zhang, Zhang et al. 2014); or Wheat (Shan, Wang et al. 2013). (Sternberg, Redding et al. 2014). Single guide RNAs are typically expressed from U6 or U3 promoters in plants, such as the wheat U6 promoter (Shan, Wang et al. 2013); the rice U3 promoter (Shan, Wang et al. 2013); the maize U3 promoter (Liang, Zhang et al. 2014); or the Arabidopsis or rice U6 promoters (Jiang, Zhou et al. 2013; Shan, Wang et al. 2013; Feng, Mao et al. 2014; Jiang, Yang et al. 2014). Ribozyme processing of transcripts from Pol II transcribed genes increases the flexibility of the system (Gao and Zhao 2014).

Plant Transformation Methods.

Any of the recombinant DNA constructs provided herein can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton). Plant transformation methods for producing transgenic plants include, but are not limited to methods for: Alfalfa as described in U.S. Pat. No. 7,521,600; Canola and rapeseed as described in U.S. Pat. No. 5,750,871; Cotton as described in U.S. Pat. No. 5,846,797; corn as described in U.S. Pat. No. 7,682,829. Indica rice as described in U.S. Pat. No. 6,329,571; Japonica rice as described in U.S. Pat. No. 5,591,616; wheat as described in U.S. Pat. No. 8,212,109; barley as described in U.S. Pat. No. 6,100,447; potato as described in U.S. Pat. No. 7,250,554; sugar beet as described in U.S. Pat. No. 6,531,649; and, soybean as described in U.S. Pat. No. 8,592,212. Many additional methods or modified methods for plant transformation are known to those skilled in the art for many plant species

### Invention Summary

A method of producing faster flowering for faster generation times in corn and sorghum is provided herein. In certain embodiments, a method for faster flowering in corn or sorghum plants comprises regulated or inducible expression of FT. In certain embodiments, a method for faster flowering in corn or sorghum plants comprises low level constitutive or tissue specific expression of FT. Faster flowering corn or sorghum plants comprising a transgene comprising an FT homolog, wherein said corn or sorghum plants flower faster than isogenic control plants lacking said transgene by at least three leaves in a developmental scale comprising the number of leaves produced prior to flowering, are also provided herein.

### BRIEF DESCRIPTION OF THE DRAWINGS

FIG. 1. Line drawing of genetic elements of plasmid pUbiqFT. Genetic elements: CaMV 35S Pro: Cauliflower Mosaic Virus 35S Promoter; Hsp70 int: intron of maize Hsp70 gene; Gfp-NptII: green fluorescent-neomycin phosphotransferase II fusion protein; T35S: terminator or 3′ polyadenylation region of Cauliflower Mosaic Virus; ZmUbiq1/int: Zea Mays Ubiquitin 1 (Ubi-1) promoter; FT: synthetic Flowering Time (SEQ ID No. 3); Nos3′: Nopaline Synthase: terminator or 3′ polyadenylation region; RBr: Agrobacterium Ti plasmid right border; pPZP: binary vector pPZP backbone; rrnBTIT2: E. coli ribosomal terminator from rrnB gene.

FIG. 2. Line drawing of genetic elements of plasmid piFT1. Genetic elements as in FIG. 1 description and the following elements: FMV: Figwort Mosaic Virus 34S promoter; XEV: modified inducible chimeric transcription factor from XEV system; PinII3′: terminator or 3′ polyadenylation region of potato PinII gene; OpPro: LexA operator with minimal CaMV 35S promoter; Adh1int: first intron of maize Adh1 gene;

FIG. 3. Floral structures present in FT containing corn plants. The floral structures from two independently transformed plants are shown. These were removed from corn plants with three to five leaves (not shown) that were regenerating from embryogenic tissues in 20 cm high petri dishes. The ovules and silks are designated and were nearly surrounded by wide leaf “ear husk” type structures that were dissected away for access to the ovules.

### DEFINITIONS

As used herein, the phrase “flowering” refers to formation of either male anthers or female ovules and stigma or complete (male and female) flowers formed on a plant.

As used herein, the phrase “developmental leaves” refers to the number of leaves formed prior to the time of flowering on a plant. Flowering can be the ear or tassel or head in the case of sorghum.

As used herein, the phrase “3 developmental leaves” refers to difference of three leaves in the number of leaves formed prior to the time of flowering on a first plant relative to a second plant.

As used herein, the phrase “gene” refers to a DNA genetic element that when in a cell causes transcription of the DNA into RNA. A gene typically comprises a promoter, transcribed region, and an associated RNA termination and/or polyadenylation processing region. Some genes may lack a RNA termination and/or polyadenylation region and still produce RNA.

As used herein, the phrases “commercially synthesized” or “commercially available” DNA refer to the availability of any sequence of 15 bp up to 2000 bp in length or longer from DNA synthesis companies that provide a DNA sample containing the sequence submitted to them.

As used herein, the term “F1” refers to the first progeny of two genetically or epigenetically different plants. “F2” refers to progeny from the self pollination of the F1 plant. “F3” refers to progeny from the self pollination of the F2 plant. “F4” refers to progeny from the self pollination of the F3 plant. “F5” refers to progeny from the self pollination of the F4 plant. “Fn” refers to progeny from the self pollination of the F(n−1) plant, where “n” is the number of generations starting from the initial F1 cross. Crossing to an isogenic line (backcrossing) or unrelated line (outcrossing) at any generation will also use the “Fn” notation, where “n” is the number of generations starting from the initial F1 cross.

“Homology” as used herein refers to sequence identity or similarity between a reference sequence and at least a fragment of a second sequence. Homology may be identified by any method known in the art, preferably, by using the BLAST or BLASTP or CLUSTAL Omega tool to compare a reference sequence or sequences to a single second sequence or fragment of a sequence or to a database of sequences. Homology includes alignment with a permutein of a sequence or protein such that alignment occurs in at least two blocks due to the circularization/opening at different N and C termini that occurs in a permutation of a gene or protein in a permutein. Optionally, homology has 70%, 75%, 80°/%, 85%, 90%, 95%, 990/% or 100% identity or similarity over a specified region, or, when not specified, over the entire sequence including the case of two regions for comparison to a permutein. The specified or entire sequence length is at least 50 amino acids or longer. As described below, BLAST (or BLASTP) or CLUSTAL Omega will compare sequences based upon percent identity and similarity.

As used herein “similarity” or “similar” refers to non-identical amino acids within the same group, where the groups are: aliphatic (Glycine, Alanine, Valine, Leucine, Isoleucine); Hydroxyl or Sulfur/Selenium-containing (Serine, Cysteine, Selenocysteine, Threonine, Methionine); Cyclic (Proline); Aromatic (Phenylalanine, Tyrosine, Tryptophan); Basic (Histidine, Lysine, Arginine); or Acidic and their Amides (Aspartate, Glutamate, Asparagine, Glutamine).

The terms “identical” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 29% identity, optionally 30°/%, 40%, 45%, 50%, 55%, 60%, 65%, 70%, 75%, 80%, 85%, 90%, 95%, 99% or 100% identity over a specified region, or, when not specified, over the entire sequence), when compared and aligned for maximum correspondence over a comparison window, or designated region as measured using one of the following sequence comparison algorithms or by manual alignment and visual inspection. Two examples of algorithms that are suitable for determining percent sequence identity and sequence similarity are the BLAST and BLAST 2.0 algorithms, which are described in Altschul et al. (1997) Nucleic Acids Res 25(17):3389-3402 and Altschul et al. (1990) J. Mol Biol 215(3)-403-410, respectively. The BLASTN program (for nucleotide sequences) or BLASTP program (for amino acid sequences) or CLUSTAL Omega are suitable for most alignments.

As used herein, the phrase “loss of function” refers to a diminished, partial, or complete loss of function.

The phrase “operably linked” as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter is linked such that the resulting translation product is in frame with the translational open reading frame that encodes the protein desired. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide DNA transfer and/or integration functions (i.e., site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences, homologous recombination sequences), and sequences that provide replication functions (i.e., bacterial origins of replication, autonomous replication sequences, centromeric sequences).

As used herein, the term “progeny” refers to any one of a first, second, third, or subsequent generation obtained from a parent plant if self-pollinated or from parent plants if obtained from a cross, or through any combination of selfing and crossing. Any materials of the plant, including but not limited to seeds, tissues, pollen, and cells can be used as sources of RNA or DNA for determining the status of the RNA or DNA composition of said progeny.

As used herein, the phrase “reference plant” refers to a parental plant or progenitor of a parental plant prior to epigenetic modification, but otherwise genetically the same as the candidate or test plant to which it is being compared.

As used herein, the terms “self”, “selfing”, or “selfed” refer to the process of self pollinating a plant.

As used herein, the term “transgene” or “transgenic” refers to any recombinant DNA that has been transiently introduced into a cell or stably integrated into a chromosome or minichromosome that is stably or semi-stably maintained in a host cell. In this context, sources for the recombinant DNA in the transgene include, but are not limited to, DNAs from an organism distinct from the host cell organism, species distinct from the host cell species, varieties of the same species that are either distinct varieties or identical varieties, DNA that has been subjected to any in vitro modification, in vitro synthesis, recombinant DNA, and any combination thereof. The terms transgene or transgenic include inserting or changing DNA sequences at endogenous genes to alter their expression or function through any non-natural process.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### EXAMPLES

### Example 1. Design and Construction of a Synthetic FT Gene for Maize and Sorghum

The protein sequence of the corn ZCN15 FT homolog was the starting point for the design of a new FT protein designed to function in corn or sorghum. The maize ZCN15 and a rice FT (SEQ ID No. 1) protein sequences were aligned by BLASTP and non-conservative amino acid changes present in ZCN15 were changed to the rice amino acid at that position. The resulting synthetic corn FT protein sequence is SEQ ID No. 2.

### Example 2. Design and Construction of a Synthetic DNA Encoding Synthetic FT Gene for Maize

A synthetic nucleic acid encoding the corn synthetic FT protein of Example 1 was designed by reverse translating a protein sequence to the possible codons encoding each amino acid at each position and then picking codons that are enriched in maize genes. The resulting synthetic DNA encoding synthetic corn is SEQ ID No. 3. Other alternative codons could be used to in the design of nucleic acid coding regions to encode the same protein.

### Example 3. Moderate to Strong Expression of Synthetic FT Prevents Plant Regeneration in Embryogenic Corn Cells

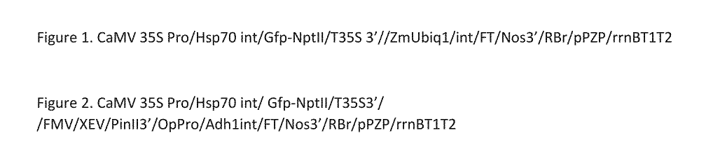

A plasmid construct pUbiqFT using the maize ubiquitin promoter with its intron to express the synthetic FT coding region (SEQ ID No. 3) and a selectable marker for corn transformation were made (FIG. 1). This plasmid was transformed into immature B104 corn embryos via Agrobacterium mediated transformation and G418 selection for GFP-NptII calli, and transgenic embryogenic callus were obtained. These embryogenic calli were unable to produce transgenic corn plants in the regeneration protocol. We concluded constitutive moderate to high levels of FT interfere with plant regeneration.

### Example 4. Low Constitutive and Inducible Expression of Synthetic Corn FT in Corn

An inducible FT expression vector was made using a modification of the XVE estradiol inducible gene expression system (see U.S. Pat. No. 6,784,340 and Zuo et al., The Plant Journal (2000) 24(2), 265-273). A plasmid map of piFT1 using the modified XVE system used here to express the FT coding region of Example 2 is shown in FIG. 2 and the sequence of the genes in piFT1 are in SEQ ID No. 4. This piFT1 plasmid was transformed into immature B104 corn embryos via Agrobacterium mediated transformation and G418 selection for GFP-NptII calli, and transgenic embryogenic callus obtained. These embryogenic calli were able to regenerate to transgenic corn shoots with roots in the regeneration protocol.

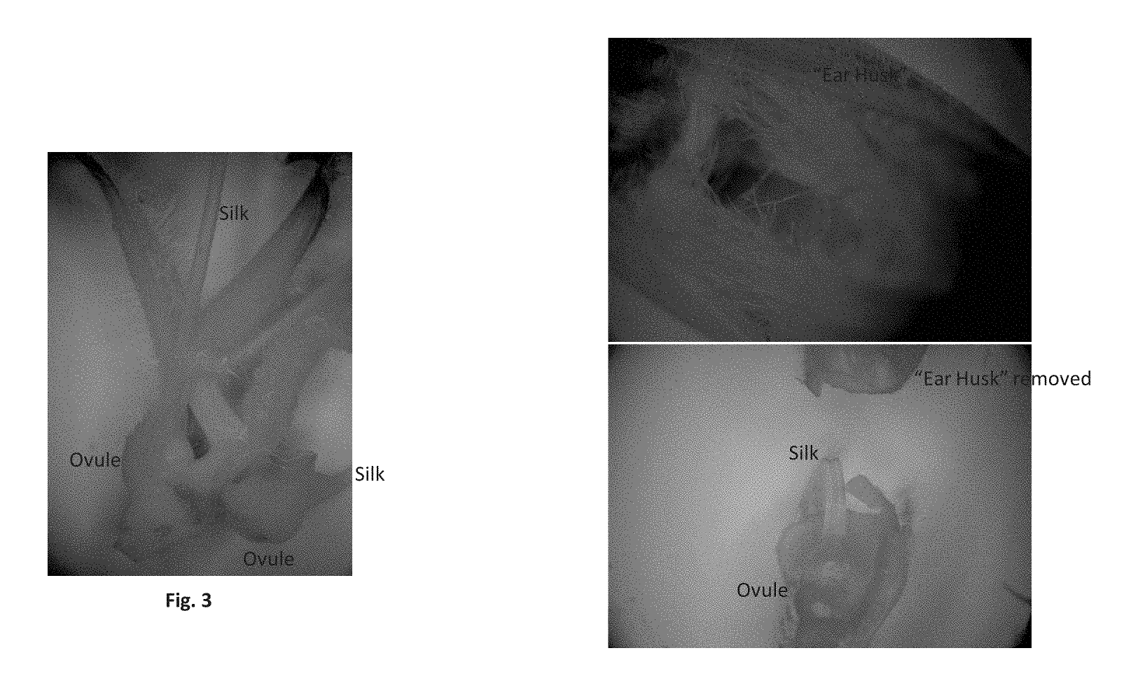

Two independently transformed regenerating transgenic plants formed flower reproductive structures while still in petri dishes while the shoots were in the three to five leaf stage. Ovules on regenerating shoots in culture have not been observed before in any maize transformations in our experience. Observation of two independent flowering events in culture amongst 42 non-induced transformation events is therefore highly significant. Dissection of these reproductive structures indicated fully formed ovules with silks were formed (FIG. 3). This early flowering is due to leaky FT expression in these particular transgenic events as estradiol inducer was not applied to these cultures. The other independent transgenic plants did not have reproductive structures and were transplanted to soil. This result demonstrates low level expression of non-native FT in corn plants is sufficient to induce very early flowering in plants having as few as three to five leaves. The inducible system for expressing FT allows the timing of this flowering to be controlled.

### Example 5. FT Induction Using the AlcR Alcohol Inducible System

A plasmid construct of the basic design of the modified XVE system of Example 4, except the AlcR chimeric transcription factor is substituted for the modified XVE, is used. The A1cR operators are substituted for the LexA operators to have constructs similar to the AlcR system as described (U.S. Pat. No. 6,605,754 and Roslan et al., The Plant Journal (2001) 28(2), 225-235). The promoter to express the AlcR chimeric transcription factor is the full length inducible rice OsSUT1 promoter (U.S. Pat. No. 7,186,821), a phloem specific promoter. Additional suitable maize active phloem promoters included but are not limited to the group consisting of rice Rpp16 and Rpp17 (Asano et al., Plant Cell Physiol. 2002 June; 43(6):668-74); rice OsABCC1; Arabidopsis AtSUC2; and Arabidopsis AtPP2-A1 (accession no. At4g19840).

Transgenic regenerable calli are obtained to produce transgenic plants in soil. These transgenic plants are root drenched with a 2% solution of alcohol to induce FT expression. The induced plants flower between the five leaf and 15^{th} leaf of development, depending on how early and often an alcohol drench is applied to the roots of the young plants.

### Example 6. Inducible Expression of FT in Maize Causes Early Flowering in Maize Plants

Transgenic plants from embryogenic callus and young plants from Example 4 were grown to maturity in the greenhouse to obtain T1 or T2 seeds to test for early flowering. T1 or T2 seeds were germinated in the presence or absence of estradiol or diethylstilbestrol (DES) to induce FT expression in a beakers with wet paper towels to induce FT expression. One week old seedlings were transplanted to soil and sprayed with induced on alternate days for another week. Plants were then maintained normally in the greenhouse or indoor growth room and observed for flowering phenotypes. Depending on the transgenic line, non-induced plants were normal, had slightly early flowering, or some leaky expressors flowered in 3 to 4 weeks after germination.

Three independently transformed plant lines that had normal to slightly early flowering times when not induced were chosen for more detailed examination. These lines flowered in 3 to 4 weeks when induced, and had normal to early flowering times when not induced. Ovule (silk) production on the tassel structure (tassel seed phenotype) was observed first as early as three weeks on induced plants. The earliest flowering plants were pollinated 27 days after imbibing seeds in the presence of inducer (estradiol or DES) and had large well developed kernels by 39 days post seed imbibition. These seeds matured and were viable when germinated, demonstrating these early flowering plants were fertile and produced viable seed at much earlier times than control plants. Anther and pollen development were not quite as fast, with the first viable pollen appearing 38 days post germination. Control plants of the same genotype flowered in about 55 to 60 days in this experiment. This system has clear benefits for accelerated generation times. We note early flowering plants were obtained from either low constitutive levels of FT expression or when induced, as both types of constitutive (non-induced) or inducible expression plants were recovered in independent transformation events.

1. A method of producing corn or sorghum plants with earlier flowering times comprising:
a. transferring a FT gene into a plant or plant cell to produce a maize or sorghum plant comprising a non-native FT gene or protein;
b. expressing said FT gene or protein of step (a) in one or more embryos, seeds or seedlings to induce early flowering, wherein said early flowering occurs at least 3 developmental leaves earlier than isogenic control plants lacking said FT gene or protein.

2. The method of claim 1, wherein expression of the FT gene or protein in step (b) is inducible.

3. The method of claim 2, wherein expression of the FT gene in step (b) is chemically inducible with a ligand that binds the ligand binding-activation domain of an ecdysone receptor, wherein the ligand is selected from the group consisting of methoxyfenozide, tebufenozide, and other compounds.

4. The method of claim 2, wherein expression of the FT gene in step (b) is chemically inducible with a ligand that binds the ligand binding-activation domain of chimeric transcription factor, wherein the ligand is selected from the group consisting tetracycline, estradiol, dexamethasone, alcohol, copper, zinc, or cadmium.

5. The method of claim 1, wherein expression of the FT gene or protein in step (b) is constitutively expressed from a weak constitutive promoter.

6. The method of claim 1, wherein expression of the FT gene or protein in step (b) is expressed from a phloem active promoter expressed at higher levels in a plant than in embryogenic callus.

7. The method of claim 1, wherein the FT gene in step (b) encodes a FT protein with at least 85%, 90%, or 95% homology to the sequence of SEQ ID No. 2.

8. The method of claim 1, wherein the FT gene in step (b) encodes a FT protein that is more homologous to SEQ ID No. 2 than to ZCN8 in the in the C-terminal region comprising 50% of the proteins.

9. The method of claim 1, wherein the FT gene in step (b) encodes a FT protein permutein.

10. The method of claim 1, wherein expression of an endogenous FT gene is altered by gene editing to form a non-native FT gene sequence comprising an altered promoter that causes early flowering relative to control non-altered parental plants.

11. A corn or sorghum plant or progeny thereof produced by the method of claim 1.
